(19) United States
(12) Patent Application Publication
(10) Pub. No.: US 2004/0138521 A1
(43) Pub. Date: Jul. 15, 2004

(54) Myocardial constraint

(76) Inventors: James R. Grabek, Minneapolis, MN (US); Michael Hoey, Shoreview, MN (US)

Correspondence Address: Beck & Tysver, P.L.L.C., Suite 100, 2900 Thomas Avenue S., Minneapolis, MN, 55416, US

(21) Appl. No.: 10/340,232

(22) Filed: Jan. 10, 2003

(51) Int. Cl. A61F 2/00; A61F 13/00

(52) U.S. Cl. 600/37

(57) ABSTRACT

A method and apparatus for treating congestive heart failure by converting the pericardium into a myocardial constraint is described along with techniques for achieving the goal.

### FIELD OF INVENTION

The present invention relates generally to the treatment of congestive heart failure by constraining the motion of the ventricles of the heart.

### BACKGROUND OF THE INVENTION

Congestive heart failure is the name given to a collection of symptoms that describe a progressive inability of the heart to pump an adequate amount of blood throughout the body. The clinical symptoms include enlargement of the heart and edema along with certain abnormalities in the pressure time history of the cardiac chambers. The disease is graded into four classes. The low-grade disease is treated with drug therapy the higher disease orders are being treated with newer and more invasive therapies.

Several clinical approaches have been proposed for the treatment of high order congestive heart failure. These include bi-ventricular pacing where the ventricles of the heart are both stimulated by pacing electrodes. Another treatment involves the application of a sock or physical constraint around the exterior of the heart to prevent further enlargement of the heart. An example of this approach is known from U.S. Pat. No. 5,702,343 to Alferness.

It has also been proposed to constrain heart motion by placing tethers and anchors between heart chamber walls so that the length of the tether limits the motion of the heart.

Although it is widely recognized that constraint of the heart is a desirable result, most mechanical constraint systems involve open chest surgery which is extremely invasive and which is typically contra-indicated for a congestive heart failure patient. For these reasons there are continuing needs to develop both mechanical constraints and the methods of applying them.

### SUMMARY OF THE INVENTION

In accordance with the present invention the heart is accessed through the pericardial space in a minimally invasive manner.

In one embodiment the pericardium itself is attached to the myocardial surface and converted into a mechanical constraint confining the motion of the heart.

In a second embodiment a mechanical constraint is deployed around the heart through the pericardial space and positioned between the pericardium and the heart.

In a third embodiment a mechanical constraint is formed within the pericardial space.

In each of these embodiments the benefit to the patient is the delivery of, or the formation of, a mechanical constraint without the need for open chest surgery.

### BRIEF DESCRIPTION OF THE DRAWINGS

Throughout the several figures identical reference numerals indicate identical structure wherein:

FIG. 1 is a schematic diagram showing access to the pericardial space through a minimally invasive process;

FIG. 2 is a schematic diagram showing the implantation of a sock like constraint;

FIG. 3 is a schematic diagram showing the application of a constraining band;

FIG. 4 is a schematic diagram showing the use of a mechanical device to injure the pericardium in a controlled fashion

FIG. 5 is a schematic diagram showing the delivery of a particulate material and the injection of an irritant or drug into the pericardial space.

### DETAILED DESCRIPTION

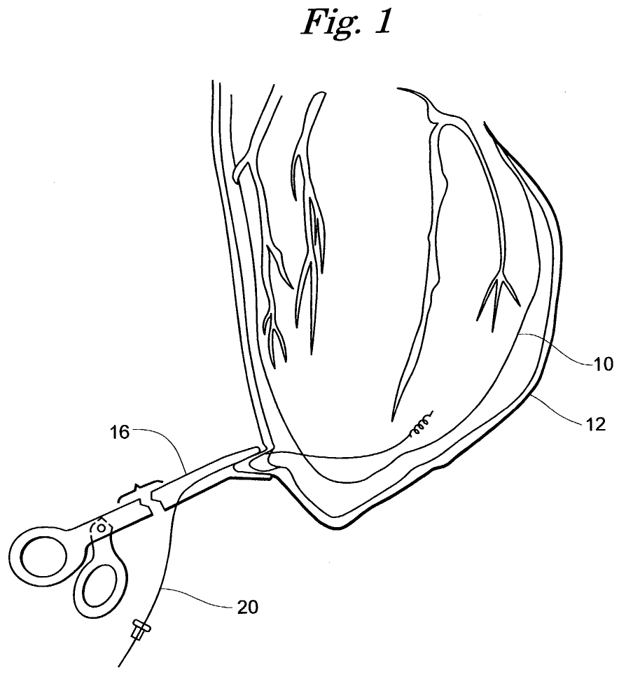

FIG. 1 shows a heart 10 and its associated pericardium 12. The surface of the patient is punctured and a pericardial access device such as the “grabber” device 16 is inserted through the incision and maneuvered under the ribs and sternum to contact the pericardium 12. As explained in U.S. Pat. No. 5,681,278 and 5,931,810 among others, a “bleb” is formed of pericardial tissue and a guidewire 20 or the like may be inserted into the pericardial space. The “Perducer” or equivalent “grabber” device 16 may be deployed with a sheath 22 or the like as illustrated in later figures.

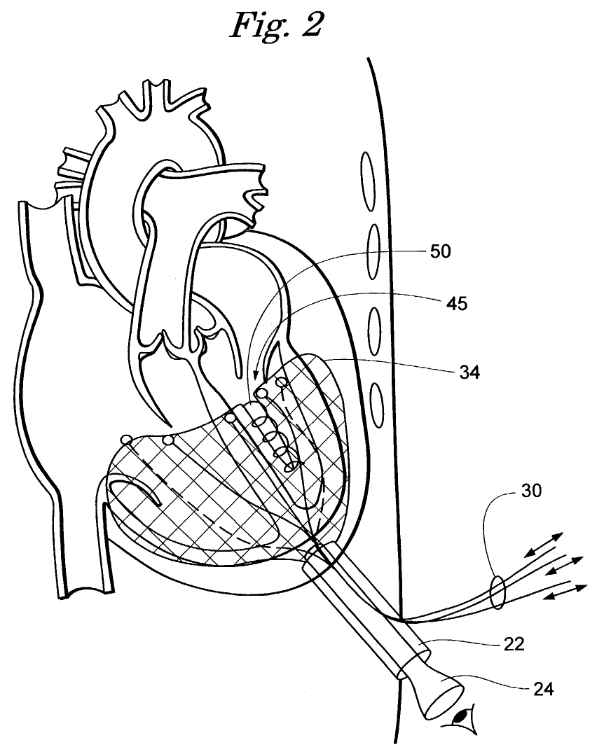

Once the pericardium is accessed, the guidewire 20 may be used alone or with the sheath 22, to introduce additional devices into the pericardial space. In FIG. 2 the pericardial space is shown in an “inflated” condition where it has moved away from the surface of the myocardium. The spacing depicted in the figure is exaggerated for purposes of clarity. In general it will be preferable to both inflate the pericardium with a fluid or gas and to position an endoscope to view the heart. Although introduction of a gas or fluid into the pericardial space along with an endoscopic observation instrument are preferred other visualization techniques are acceptable for carrying out the invention.

In FIG. 2 the pericardial access device has been removed and a sheath 22 and scope 24 have been introduced. In the figure the guidewire 20 has been replaced with a multi-armed application tool 30 that is attached to a mesh membrane 34. In use the physician advances the mesh membrane 34 over the apex of the heart. This motion positions the mesh 34 around the circumference of the ventricular chambers of the heart.

In operation, the beating heart, along with the applicator tool 30 cooperates to advance the mesh membrane over the apex of the heart and on to the ventricular walls. It is preferred to have independent control over each arm of the applicator tool 30. The multiple arms may be moved together or they may be advanced sequentially. Once placed the mesh 34 may be released from the multi-arm tool 30 and sutured, glued or otherwise affixed to the myocardial surface. Next and the scope 24 and introduction tools 22 removed. Deflation of the pericardial sac 12 will collapse the pericardium around the implanted mesh 34. Also seen in FIG. 2 is the deployment of a sac-like mesh membrane or sock 34 around the ventricles with an open section 45 closed by a “lace” structure 50. After encircling the ventricles the “lace” 50 may be tightened to fit the “sock” to the heart 10. Although direct connection of the mesh to the epicardial surface of the heart is preferred the mesh 34 may be left “floating” in position and the collapsed pericardium will trap the mesh 34 in position around the heart. A wide variety of materials may be used for the mesh sock 34 including Teflon or Dacron or ntinol braid. It is preferred to use non-resorbable materials as the strength or integrity of the sock is relied on to constrain the heart.

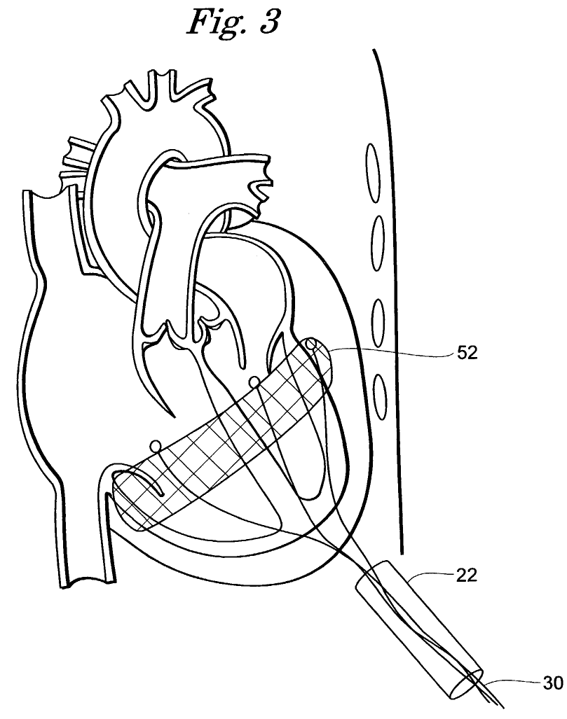

FIG. 3 shows the multi-arm tool 30 delivering a single mesh band 52 to the pericardial space around the ventricles. The mesh band 52 may be made of any of several exemplary biocompatible materials including Dacron and Teflon mesh materials. Biodegradable or resorbable materials are also acceptable including polymers made of poylactate and the like. In these embodiments it is expected that the pericardium will scar and adhere to the eipcardial surface of the heart. The relatively inelastic scar tissue will provide the mechanical strength to constrain the motion of the heart. The band that encircles the heart will localize the mechanical strength to the meridian of the heart and this ability to localize the structure may prove to be an advantage. It must be understood that the band may be extended to fully enshroud the heart if desired. The resorbable mesh may be desirable because it “disappears” after the scar formation and is only acutely present in the therapy.

In the FIG. 2 and FIG. 3 deployments the multi-arm tool is released form the implanted device 34 or 52 and removed from the pericardial sac. Various release feathers may be used to detach the mesh structures from the arms the simplest expedient is to all the independent arms to rotate about their axes to unhook from the mesh. Although individually and independently movable arms are preferred the arms may be attached to one another so that they move as a group. A switch able version may also be employed where the connection between the arms is selectable. Thus independent arm, coupled arm and selectable arm embodiments are contemplated within the scope of the invention.

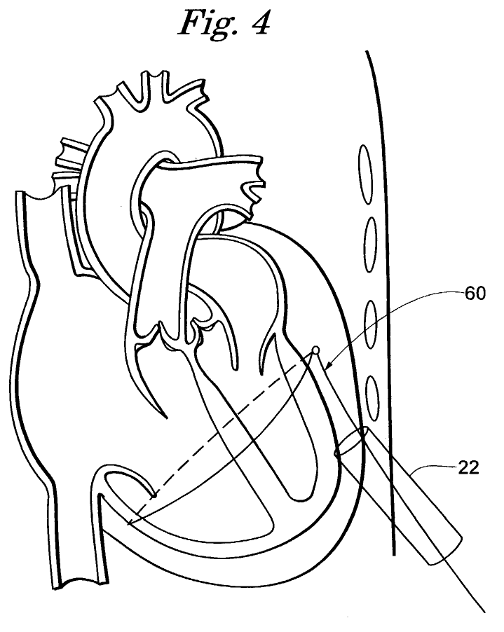

In FIG. 4 a large snare-like structure 60 is being manipulated around the heart to injure the myocardial surface and the surface of the pericardium to permit scarring. It is anticipated that scarring will cause the pericardium itself to adhere to the myocardial surface forming a constraint device to treat congestive heart failure. The multi arm tool 30 without the mesh is an alternative tool for abrading the tissues. It is also anticipated that n abrasive mesh can be substituted for the mesh 34 and this structure used for abrading the tissues. In this alternate embodiment the abrasive mesh would be permanently attached to the multi arm tool 30.

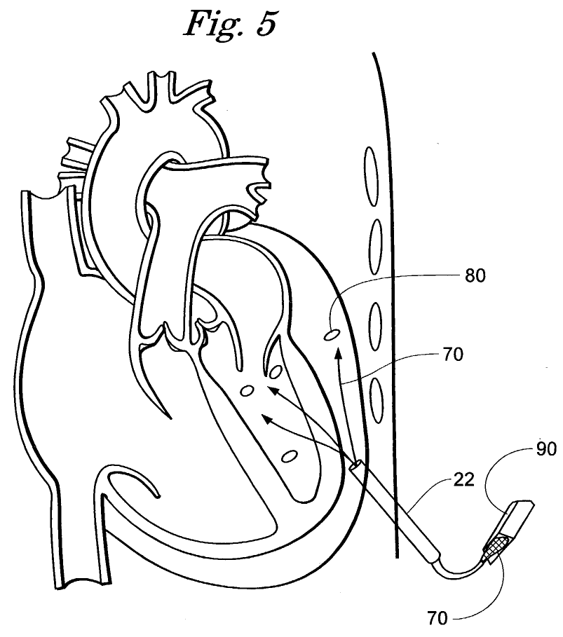

In FIG. 5 a drug 70 of known and controlled toxicity is being introduced into the pericardial space with the syringe 90, to cause a sufficient injury to encourage the formation and adhesion of the pericardium to the surface of the heart. Streptomycin and other antibiotics are known to create injury when administered into the pericardium.

Also in FIG. 5 particles or beads 80 that may or may not be drug coated are being introduced into the pericardial space along with the drug 70 to injure the myocardium and pericardium to enhance adhesions formed between the two structures. Talc is known to induce scarring of the pericardium and it is an example of a particle 80 material. It is expected that the motion of the heart will cause the particles to accumulate in particular circumferential locations around the ventricles providing a more localized injury or drug delivery. It is anticipated that the particles may congeal and polymerize to form a band or mechanical constraint around the heart inside the pericardial space.

What is claimed is:

1. A method of constraining the heart comprising:
accessing the pericardial space;
inserting a mesh device into the pericardial space that conforms to the heart and adheres the pericardial sac to the myocardial surface.

2. The method of claim 1 wherein the inserting step comprises:
coupling a mesh device to the free arms of a multi arm tool;
inserting the mesh and tool through a sheath positioned in the pericardial space;
advancing the mesh over the apex of the heart; thereby expanding the mesh into contact with the heart;
releasing the mesh from the arms and, removing the tool.

3. The method of claim 1 wherein the accessing step comprises:
forming a bleb of pericardial tissue;
inserting a guidewire into the pericardial space;
advancing the guidewire over the apex of the heart;
delivering a sheath defining a passageway, over the guidewire; thereby permitting the delivery of the tool as set forth in claim 1.

4. A method of constraining the heart comprising:
accessing the pericardial space;
inserting a mesh device into the pericardial space;
moving the device to abrade tissue over selected portion of the epicardial surface; thereby promoting the formation of scar tissue thereby, adhering the pericardial sac to the myocardial surface.

4. A method of constraining the heart comprising:
accessing the pericardial space;
inserting a tubular device into the pericardial space;
injecting an irritant into the pericardial sac; thereby promoting the formation of scar tissue thereby, adhering the pericardial sac to the myocardial surface.

5. The method of claim 4 wherein said irritant is a non-resorbable particulate.

6. The method of claim 4 wherein said irritant is a resorbable particulate formed from polylactaic acid.

7. The method of claim 4 wherein said irritant is an antibiotic drug.

8. A device for constraining the motion of the heart comprising:
a mesh band for encircling the heart.

9. The device of claim 8 wherein said mesh band is made of a material selected from the group; Dacron, Teflon, Nitinol, Cotton.
